(19) United States
(12) Patent Application Publication
(10) Pub. No.: US 2022/0244237 A1
(43) Pub. Date: Aug. 4, 2022

(54) SYSTEM AND DEVICE FOR SEPARATING OR MIXING BIOLOGICAL FLUIDS AND/OR TISSUE

(71) Applicant: Aesthetics Biomedical, Inc., Phoenix, AZ (US)

(72) Inventor: Shaun Wootten, Phoenix, AZ (US)

(21) Appl. No.: 17/563,872

(22) Filed: Dec. 28, 2021

Related U.S. Application Data
(60) Provisional application No. 63/130,980, filed on Dec. 28, 2020.

(51) Int. Cl. G01N 33/49; B01D 21/26; A61J 1/20; G01N 1/34

(52) U.S. Cl. CPC G01N 33/491; G01N 1/34; A61J 1/2089; B01D 21/262

(57) ABSTRACT

A system and apparatus for separating biological tissue and/or biological fluids and in particular a system and apparatus for separating whole blood in order to obtain plasma or platelet-rich plasma which includes embodiments of separation vessels that are rotated about a vertical axis. Some separation vessels include both a separation chamber and a collection chamber. The invention also includes a system and apparatus for mixing biological tissue and/or biological fluids with other materials to create pharmaceutical grade injectables.

### CROSS-REFERENCE TO RELATED APPLICATIONS

This application claims priority to provisional patent application having Ser. No. 63/130,980, filed Dec. 28, 2020, which is herein incorporated by reference in its entirety.

### FIELD OF INVENTION

The present invention is directed to a system and device for separating biological tissue and/or biological fluids. More particularly, the present invention is directed to a system and device for fractionating blood in order to extract platelet rich plasma (PRP). In addition, the present invention may be used to mix biological tissue and/or biological fluids with other materials to create pharmaceutical grade aesthetic injectables such as fillers for cosmetic volume enhancement.

### BACKGROUND OF THE INVENTION

Whole blood is routinely separated into its various therapeutic components such as red blood cells, platelets, and plasma. Plasma separation from whole blood has been traditionally performed by centrifugation which typically takes 15 to 20 minutes utilizing a centrifuge which rapidly rotates containers, such as tubes, by applying centrifugal force to the blood contained in the tubes to separate the various components of the blood having different densities. The radial acceleration causes denser particles such as red blood cells to settle to the bottom of the tube while lower density particles such as plasma rise to the top. When a tube of blood is removed from a centrifuge, the components have separated into three layers: blood plasma, the huffy coat containing platelet cells, and red blood cells.

Other technologies have also been used to separate plasma from whole blood including sedimentation, fibrous and non-fibrous membrane filtration, lateral flow separation, microfluidics cross flow filtration, and other microfluidics hydrodynamic separation technologies. Still, centrifugation with a centrifuge, and typically two centrifugations, tends to be the preferred method for obtaining platelet-rich plasma for treating patients. However, centrifuges can be noisy, take up valuable benchtop space or work space, and must be balanced to ensure safe and proper use.

The system and device of the present invention for separating blood is simple, easy to operate, has a compact footprint, may be automated to eliminate user contamination, and, unlike a centrifuge, does not need to be balanced in order to be safely and, properly used. The system and device of the present invention for separating blood also eliminates the need for a rotor which is a large sized component of the centrifugation process. Eliminating the need for a rotor further reduces the space requirements of the present invention's system and device for separating blood and obtaining platelet-rich plasma.

The system and device of the present invention can be used for separating different types of biological tissue as well as separating different types of biological fluids. The system and device of the present invention may alternatively be used for mixing biological tissue and/or biological fluids with other materials such as a biopolymer gel to create pharmaceutical grade aesthetic injectables such as fillers for cosmetic volume enhancement.

### SUMMARY OF THE INVENTION

The present invention includes a system and apparatus for separating biological tissue and/or biological fluids and in particular a system and apparatus for separating whole blood in order to obtain plasma or platelet-rich plasma. The invention includes various embodiments of separation vessels which include a hollow spherical shaped separation vessel as well as separation vessels having various shapes of separation chambers connected to various shapes of collection chambers where all of the separation vessels are rotated about a vertical axis to separate the biological fluid, such as blood, into its component parts.

In one exemplary embodiment, a device for separating biological tissue and/or biological fluids includes a hollow sphere shaped vessel having an access port contained within a surface of the hollow sphere shaped vessel. The device may also include a housing for retaining the hollow sphere shaped vessel where the housing has an air inlet positioned within the housing so that air provided through the air inlet enables the hollow sphere shaped vessel to spin in a counterclockwise direction on a vertical axis. The device may also include a hollow cylindrical member extending into an interior of the hollow sphere shaped vessel such that the hollow cylindrical member is contiguous with an exterior surface of the hollow sphere shaped vessel. The housing may also include a spindle or rod member capable of engaging with the hollow cylindrical member to spin the hollow sphere shaped vessel in a clockwise direction on a vertical axis.

In another exemplary embodiment, a device for separating biological tissue and/or biological fluids includes a hollow cylindrical shaped vessel having an access port contained within a surface of the hollow cylindrical shaped vessel. The device may also include a housing for retaining the hollow cylindrical shaped vessel where the housing has an air inlet positioned within the housing so that air provided through the air inlet enables the hollow cylindrical shaped vessel to spin in a counterclockwise direction on a vertical axis. The device may also include a second smaller hollow cylindrical member extending into an interior of the hollow cylindrical shaped vessel such that the second smaller hollow cylindrical member is contiguous with an exterior surface of the hollow cylindrical shaped vessel. The housing may also include a spindle or rod member capable of engaging with the second smaller hollow cylindrical member to spin the hollow cylindrical shaped vessel in a clockwise direction on a vertical axis.

In yet another exemplary embodiment, a separation vessel for separating biological tissue and/or biological fluids includes a hollow hockey puck shaped separation chamber having a circular shaped opening in a bottom of the separation chamber and a hollow tubular shaped collection chamber having a top open end where the top open end of the collection chamber is in communication with the circular shaped opening in the bottom of the separation chamber. The separation vessel may further include a moveable or retractable cover member that covers the circular shaped opening in the bottom of the hollow hockey puck shaped separation chamber. The separation vessel may also include an access port contained within a surface of the hollow hockey puck shaped separation chamber.

In still another exemplary embodiment, a separation vessel for separating biological tissue and/or biological fluids includes a hollow donut-shaped/ring-shaped separation chamber having a ring shaped opening contained in a bottom of the separation vessel and a hollow tubular-shaped collection chamber having an open top end where the open top end of the collection chamber is in communication with the ring shaped opening in the separation chamber. The separation vessel may further include an access port contained within a surface of the hollow donut-shaped/ring-shaped separation vessel. The hollow tubular-shaped collection chamber may include a closed funnel shaped bottom having an access port. In addition, the closed funnel shaped bottom of the tubular-shaped collection chamber may be removeable.

The present invention also includes a method and apparatus for separating biological tissue and/or biological fluids which utilizes the previously described separation vessels. In one exemplary embodiment, the apparatus for separating biological tissue and/or biological fluids includes a housing, a spindle/rod member contained within the housing, a motor contained within the housing that is capable of rotating the spindle/rod member, and a separation vessel having an upper hollow separation chamber in communication with a lower hollow tubular-shaped collection chamber where the spindle/rod member is capable of engaging with the upper hollow separation chamber of the separation vessel to rotate/spin the separation vessel about a vertical axis. The separation apparatus may further include a user interface to select at least a rotation speed and/or time for rotating/spinning the separation vessel. The separation apparatus may also include a biohazard collection bin contained within the housing and a ramp/pathway contained within the housing for directing used separation chambers of the separation vessels into the biohazard collection bin. In addition, the separation apparatus may include an adjustable tray for retaining a container for containing a separated biological fluid and/or an ultraviolet light source contained within the housing for exposing separated components of a biological fluid contained within the separation chamber to ultraviolet light.

In addition, the present invention includes a system and apparatus for mixing biological tissue and/or biological fluids with other materials utilizing the same device embodiments described above that are used to separate biological tissue and/or biological fluids. The present invention also includes a method and apparatus for mixing biological tissue and/or biological fluids with other materials that utilizes the same system and apparatus described above that are used to separate biological tissue and/or biological fluids.

### BRIEF DESCRIPTION OF THE DRAWINGS

FIG. 1 is a schematic showing a first exemplary embodiment of a separation vessel of the present invention for separating biological fluids such as whole blood and the forces used to rotate the separation vessel;

FIG. 2 is a perspective view of the first exemplary embodiment of the separation vessel shown in FIG. 1;

FIG. 3 is a bottom perspective view of a second exemplary embodiment of a separation vessel of the present invention for separating biological fluids such as whole blood using the forces shown in FIG. 1 to rotate the separation vessel;

FIG. 4 is a bottom perspective view of the second exemplary embodiment of the separation vessel shown in FIG. 3 shown along with a syringe used for extracting plasma or platelet-rich plasma from the separation vessel once the whole blood is separated;

FIG. 5 is a perspective view of a third exemplary embodiment of a separation vessel of the present invention for separating biological fluids such as whole blood using the forces shown in FIG. 1 to rotate the separation vessel;

FIG. 6 is a schematic showing a top view of a fourth exemplary embodiment of a separation vessel of the present invention for separating biological fluids such as whole blood and the forces used to rotate the separation vessel;

FIG. 7 is a top perspective view of the fourth exemplary embodiment of the separation vessel shown in FIG. 6 with locations of separated, components of whole blood in the separation chamber after separation;

FIG. 8 is a schematic showing a perspective view of an exemplary locking mechanism used to lock the tubular shaped collection chamber of the separation vessel to the donut shaped/ring shaped separation chamber of the separation vessel of the fourth exemplary embodiment of the separation vessel shown in FIG. 7;

FIG. 9 is a perspective view of the tubular shaped collection chamber of the separation vessel of the fourth exemplary embodiment of the separation vessel shown in FIG. 7;

FIG. 10 is a perspective view of a second exemplary embodiment of the tubular shaped collection chamber of the separation vessel of the fourth exemplary embodiment of the separation vessel shown in FIG. 7;

FIG. 11 is a perspective view of the second exemplary embodiment of the tubular shaped collection chamber of the separation vessel shown in FIG. 10 along with a syringe used for extracting platelet-rich plasma from the tubular shaped collection chamber of the separation vessel;

FIG. 12 is a perspective view of a third exemplary embodiment of the tubular shaped collection chamber of the separation vessel of the fourth exemplary embodiment of the separation vessel shown in FIG. 7;

FIG. 13 is a vertical cross-sectional view of the fourth exemplary embodiment of the separation vessel shown in FIG. 7 with the second exemplary embodiment of the tubular shaped, collection chamber shown in FIGS. 10 and 11 along with indications of where the separated fractions of blood would exist after separation of a whole blood sample utilizing the hollow donut-shaped/ring-shaped separation chamber shown in FIG. 7;

FIG. 14 is the same vertical cross-sectional view shown in FIG. 13 shown after the plasma portion of the separated whole blood sample falls into the tubular shaped collection chamber so that it can be extracted from the tubular shaped collection chamber with a syringe;

FIG. 15 is a top plan view showing the inside of the hollow donut-shaped/ring-shaped separation chamber depicted in FIG. 13 and the locations of the separated components of a whole blood sample after the whole blood sample has been separated using the hollow donut-shaped/ring-shaped separation chamber;

FIG. 16 is a perspective view of a fifth exemplary embodiment of a separation vessel of the present invention for separating biological fluids such as whole blood into its component parts;

FIG. 17 is a vertical cross-sectional view of the fifth exemplary embodiment of the separation vessel of the present invention shown in FIG. 16;

FIG. 18 is a top plan view showing the inside of the separation chamber depicted in FIG. 16 and the locations of the separated components of a whole blood sample after the whole blood sample has been separated using the separation chamber; and

FIG. 19 is a cross-sectional view of a system/apparatus and its components for separating biological fluids such as whole blood which utilizes the fourth exemplary embodiment of the separation vessel shown in FIG. 7 shown along with a perspective view of part of the tubular portion of the separation vessel and a container for collecting platelet rich plasma from the tubular portion of the separation vessel seated on an adjustable tray.

### DESCRIPTION OF EXEMPLARY EMBODIMENTS

The exemplary embodiments of the present invention's system and device for separating biological tissue and/or biological fluids, and especially for separating whole blood into its component parts including platelet-rich plasma, are simple, quiet, low-cost, compact, fast, and easy to operate. The system includes a housing capable of containing multiple components including components capable of rotating alternative embodiments of a separation vessel. Alternative embodiments of the separation vessel include a hollow sphere shaped vessel, a hollow donut-shaped/ring-shaped separation chamber capable of being connected to a hollow tubular-shaped collection chamber, and a hockey puck-shaped separation chamber also capable of being connected to a hollow tubular-shaped collection chamber.

The identity of the elements/features that relate to the numbers shown in the drawing figures are as follows:

10 hollow sphere shaped separation vessel

12 female Luer-Lock fitting (that mates with male Luer-Lock fitting on syringe)

20 hollow sphere shaped separation vessel

22 female Luer-Lock fitting (that mates with male Luer-Lock fitting on syringe)

24 hollow cylindrical member

26 open end (of hollow cylindrical member 24)

28 closed end (of hollow cylindrical member 24)

29 syringe (having male Luer-Lock fitting)

30 hollow cylindrical shaped separation vessel

32 female Luer-Lock fitting (that mates with male Luer-lock fitting on syringe)

34 smaller hollow cylindrical member

36 open end (of smaller hollow cylindrical member 34)

38 closed end (of smaller hollow cylindrical member 34)

40 separation vessel

42 hollow donut-shaped/ring-shaped separation chamber (of separation vessel 40)

44 top (of hollow donut-shaped/ring-shaped separation chamber 42)

46 bottom (of hollow donut-shaped/ring-shaped separation chamber 42)

48 short cylindrical shaped channel (in middle of hollow donut-shaped/ring-shaped separation chamber 42)

50 top open end (of short cylindrical shaped channel 48, capable of engaging with a spinning rod/spindle that spins/rotates the separation vessel 40)

52 bottom open end (of short cylindrical shaped channel 48)

53 ring-shaped opening (in bottom 46 of hollow donut-shaped/ring-shaped separation chamber 42 that surrounds short cylindrical shaped channel 48 in middle of hollow donut-shaped/ring-shaped separation chamber 42)

54 hollow tubular-shaped collection chamber (of separation vessel 40)

56 open top end (of hollow tubular-shaped collection chamber 54 capable of engaging with ring-shaped opening 53)

58 open bottom end (of hollow tubular shaped collection chamber 54)

60 rotatable locking mechanism

62 plurality of hollow notch members (extending from circumference of ring-shaped opening 53 in bottom 46 of hollow donut-shaped/ring-shaped separation chamber 42)

63 plurality of solid notch members (extending from circumference of open top end 56 of hollow tubular-shaped collection chamber 54)

65 female Luer-Lock fitting (that mates with male Luer-Lock fitting on syringe)

74 hollow tubular-shaped collection chamber

76 open top end (of hollow tubular-shaped collection chamber 74 capable of engaging with ring-shaped opening 53)

78 closed funnel-shaped bottom end

80 female Luer-Lock fitting (that mates with male Luer-Lock fitting on syringe)

81 syringe

84 hollow tubular-shaped collection chamber

86 open top end (of hollow tubular-shaped collection chamber 84)

88 open bottom end (of hollow tubular-shaped collection chamber 84)

90 hollow funnel-shaped member

92 open top end (of hollow funnel-shaped member 90)

94 closed bottom end (of hollow funnel-shaped member 90)

96 female Luer-lock fitting (that mates with male Luer-Lock fitting on syringe)

100 rotatable locking mechanism

102 plurality of hollow notch members (extending from circumference of open bottom end 88)

104 plurality of solid notch members (extending from open top end 92)

140 separation vessel

142 hockey puck shaped separation chamber

144 top (of hockey puck shaped separation chamber 142)

146 bottom (of hockey puck shaped separation chamber 142)

148 circular shaped opening (in bottom 146 of hockey puck shaped separation chamber 142)

149 moveable/retractable cover member (which covers circular shaped opening 148)

150 female Luer-Lock fitting

154 hollow tubular-shaped collection chamber

156 open top end (of hollow tubular shaped collection chamber 154 capable of engaging with circular shaped opening 148)

158 open bottom end (of hollow tubular shaped collection chamber 154)

200 separation system/apparatus

202 housing

204 motor

206 spindle/rod (for rotating separation vessel)

400 separation vessel

402 hollow separation chamber

454 hollow tubular-shaped collection chamber

208 printed circuit board

210 electrical cord (for connection to power source)

212 actuator (for vertical movement and release of hollow separation chamber 402)

214 ramp/pathway (for directing movement of hollow separation chamber 402)

216 biohazard collection bin (for used hollow separation chambers 402)

218 adjustable tray

220 container for plasma/PRP

222 laser to read radio-frequency identification (RFID)

224 user interface (to program rotation speed of separation vessel 400, time for rotating/spinning of separation vessel 400, etc.)

226 UV light source

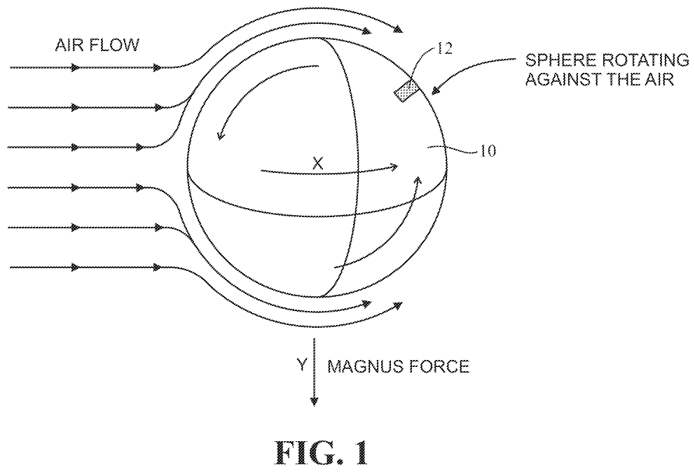

FIG. 1 is a schematic showing a first exemplary embodiment of a separation vessel 10 of the present invention for separating biological fluids such as whole blood and the forces used to rotate the separation vessel. Separation vessel 10 has a hollow spherical shape with a female Luer-Lock fitting 12 positioned within a surface of the separation vessel 10 to enable a male Luer-Lock fitting on a syringe to lock with the female Luer-Lock fitting 12 to provide a leak-free connection between the syringe and the separation vessel 12. The Luer-taper is a standardized system of small-scale fluid fittings used for making leak-proof or leak-free connections between a male-taper fitting and its mating female part on medical instruments such as syringe tips and needles. Luer-Lock is a proprietary name for a specific Luer taper made by Beckton Dickenson but other types of Luer tapers may be utilized with the present invention. In order to utilize the separation vessel 10 of the present invention, blood can be drawn from a patient using a needle connected to a syringe. The needle can then be removed from the syringe and the syringe containing the patient's blood can be deposited into the separation vessel by connecting the male Luer-Lock fitting on the syringe to the female Luer-lock fitting 12 on the separation vessel 10 and pushing the blood contents of the syringe into the separation vessel 10. Alternatively, a leak-proof rubber member, like the rubber closure contained on vials of liquid where the liquid is withdrawn using a syringe, may be positioned on a surface of the separation vessel 10. The needle of a syringe containing a patients blood can then pierce through the rubber member and expel the blood from the syringe into the separation vessel 10.

Another alternative method for depositing blood within the separation vessel 10 may include having separation vessel 10 under vacuum so that a Vacutainer type double ended needle along with a needle holder that conforms to the shape of the separation vessel 10 can be used to pull blood from a patient through the needle and into the separation vessel 10 using the vacuum in the separation vessel 10. Separation vessel 10 may be included as part of a standard blood collection kit and/or along with a Vacutainer type double needle and needle holder that conforms to the shape of the separation vessel 10.

Separation vessel 10 may be prefilled with a predetermined amount of one or more of the following: an anticoagulant, a hyaluronic acid, a selector gel including but not limited to a thixotropic gel, a biopolymer gel, a cell encapsulation ingredient (e.g. liposome, micelle) cell buffer, an anti-platelet effect buffer, an anti-microbial, an anti-bacterial, and a cell activation fluid. The cell activation fluid may include one or more of an autologous thrombin, an allogeneic thrombin, a bovine thrombin, a calcium ion, a calcium chloride, a calcium carbonate, and a calcium gluconate. The cell buffer may comprise one or more of the following and their derivatives: a glucose, a sodium ion, a sodium chloride, a magnesium ion, a magnesium chloride, a magnesium phosphate, a potassium ion, a potassium chloride, a potassium phosphate, a sodium gluconate, a pH regulator such as sodium hydroxide, and a sodium bicarbonate. The anti-platelet effect buffer may include any one or more of a honeysuckle, a thymol, an o-cymen-5-ol, a phenol, and a botanical extract.

Once a patient's blood is deposited within the separation vessel 10, the separation vessel 10 is spun on a vertical axis in a counterclockwise direction X so that the downward force Y separates the blood contained in the separation vessel 10 into blood components or fractions. The separation vessel 10 may alternatively be spun in a clockwise direction on a vertical axis as long as the separation vessel 10 is spinning against air flow so that the separation vessel 10 is in a topspin rotation. Separation vessel 10 may be rotated in any number of ways including, but not limited to, use of a rod on which the separation vessel 10 may be made to spin on a vertical axis via mechanical rotation of the rod with a motor, use of forced air used to spin the separation vessel 10 on a vertical axis by securing the separation vessel 10 in a specific orientation, and use of a rotating magnetic field that spins the separation vessel 10 about a vertical axis.

Magnets may be placed externally to the separation vessel 10 and reside in a system or housing where the magnets levitate the separation vessel 10 to reduce friction. Separation vessel 10 may include internal magnets and in some instances an electromagnet where the electromagnet induces an electrical current and magnetic field on to the vessel as the vessel rotates to enhance fractionation. The magnets may be placed such that they help guide the fluid through chambers that may be contained in separation vessel 10 for further delineation of a biological fluid specimen.

After separation of the blood using separation vessel 10, red blood cells will be located in the lowest pan of the separation vessel 10 followed by a buffy coat on top of the red blood cells and plasma on top of the buffy coat. The male Luer-Lock fitting on a syringe can then be connected to the female Luer-Lock fitting 12 on the separation vessel 10 to withdraw the plasma portion of the patient's separated blood from the separation vessel 10. The patient's plasma or platelet-rich plasma can then be used to treat the patient by injecting it into the patient or it can be used to create personalized cosmetic formulations for the patient. As previously discussed above, the plasma or platelet-rich plasma may also be withdrawn from the separation vessel 10 using a needle connected to a syringe where the needle pierces a rubber member on the separation vessel 10 to withdraw the plasma or platelet-rich plasma.

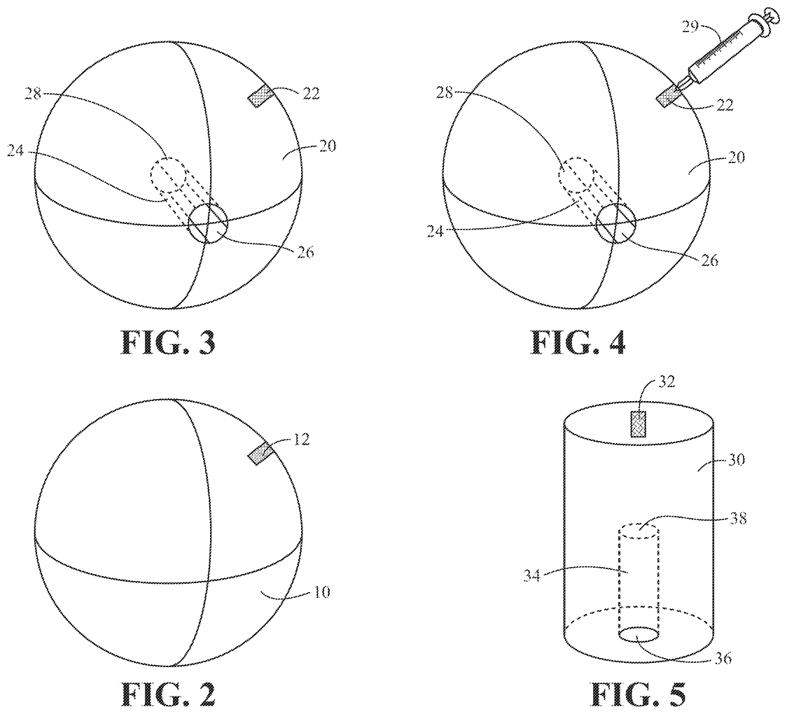

FIG. 2 is a perspective view of the first exemplary embodiment of the separation vessel shown in FIG. 1. Separation vessel 10 is a hollow sphere having a female Luer-Lock fitting 12, or similar female type Luer taper fitting, that enables connection to a male Luer-Lock type fitting on a syringe, or similar male type Luer fitting on a syringe. Once connected, a separated component of a biological fluid such as plasma or platelet-rich plasma separated from a patient's whole blood can be withdrawn from the separation vessel 10. It will be understood by those skilled in the art that any number of biological fluids that contain various components may be separated into their various components using the system and device of the present invention which includes all of its various shaped and described separation vessels.

A second exemplary embodiment of a separation vessel 20 of the present invention for separating biological fluids such as whole blood using the forces shown in FIG. 1 to rotate the separation vessel 20 is shown in FIG. 3. Separation vessel 20 is a hollow sphere shaped separation vessel that has a hollow cylindrical member 24 extending upward from a bottom of the hollow sphere shaped separation vessel 20 to a midpoint within the hollow sphere shaped separation vessel 20. The hollow cylindrical member 24 has an open end 26 contiguous with the outer surface of the hollow sphere shaped separation vessel 20 and a closed end 28 contained within the interior of the hollow sphere shaped separation vessel 20 which enables a rod or spindle to be positioned within the hollow cylindrical member 24 to spin or rotate the hollow sphere shaped separation vessel 20 about a vertical axis. The hollow sphere shaped separation vessel 20 also includes a female Luer-Lock fitting 22, or similar Luer taper type fitting, as previously described above with reference to FIG. 1. FIG. 4 is a bottom perspective view of the second exemplary embodiment of the separation vessel 20 shown in FIG. 3 shown along with a syringe 29 used for extracting plasma or platelet-rich plasma from the separation vessel 20 once the whole blood is separated. As previously mentioned above, it will be understood by those skilled in the art that other biological fluid components may be extracted with syringe 29 after utilizing hollow spherical shaped separation vessel 20 to separate a biological fluid into its various components.

FIG. 5 is a perspective view of a third exemplary embodiment of a separation vessel 30 of the present invention for separating biological fluids such as whole blood using the forces shown in FIG. 1 to rotate or spin the separation vessel 30. Separation vessel 30 is a hollow cylindrical separation vessel that has a smaller hollow cylindrical member 34 extending upward from a bottom of the hollow sphere shaped separation vessel 20 to a midpoint within the hollow cylindrical shaped separation vessel 30. The smaller hollow cylindrical member 34 has an open end 36 contiguous with the outer surface of the bottom of hollow cylindrical shaped separation vessel 30 and a closed end 38 contained within the interior of the hollow cylindrical shaped separation vessel 30 which enables a rod or spindle to be positioned within the smaller hollow cylindrical member 34 to spin or rotate the hollow cylindrical shaped separation vessel 30 about a vertical axis. The hollow cylindrical shaped separation vessel 30 also includes a female Luer-Lock fitting 32, or similar Luer taper type fitting, as previously described above with reference to FIG. 1. The female Luer-Lock fitting 32 is preferably positioned within a side of hollow cylindrical shaped member separation vessel 30 to enable most of the plasma or platelet-rich plasma separated from a patient's whole blood sample to be removed from the hollow cylindrical shaped separation vessel 30. It will be understood by those skilled in the art that the female Luer-type fitting 12, 22, 32, or alternatively a leak-proof rubber member, may be positioned anywhere on the separation vessels 10, 20, 30 to enable withdrawal or extraction of a particular separated component of a biological fluid from the interior of the separation vessel. In the case of separating plasma or platelet-rich plasma from whole blood, the female Luer-type fitting, or alternatively a leak-proof rubber member/fitting, of the separation vessel is preferably positioned within the top half of the separation chamber since the plasma or platelet-rich plasma has a lower density than other components of whole blood and will be located at the top of the separated blood components. In instances where other biological fluids are being separated using the separation vessels of the present invention, it may be preferable to position the female Luer-type fitting, or alternatively a leak-proof rubber member/fitting, in the lower half of the separation vessel or near the bottom of the separation vessel if the separated component of the biological fluid being sought has a greater density than the other components.

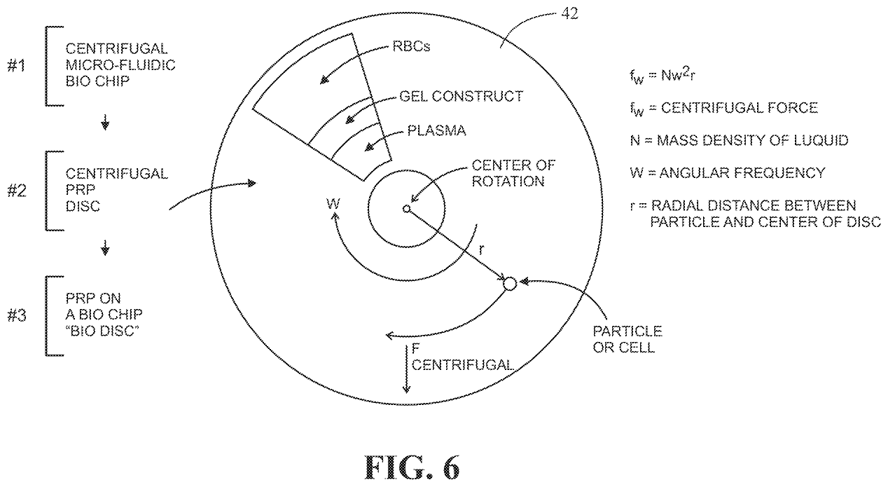

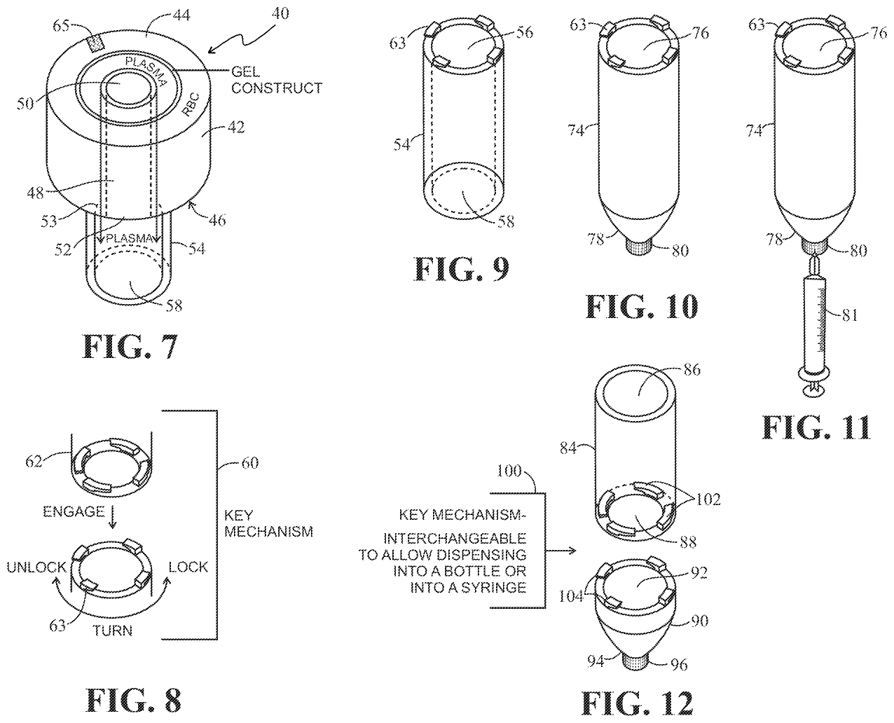

FIG. 6 is a schematic showing atop view of a fourth exemplary embodiment of a separation vessel 42 of the present invention for separating biological fluids such as whole blood and the forces used to rotate the separation vessel 42. Part of separation vessel 42 takes the form of a hollow donut-shaped/ring shaped separation chamber or a disc shaped/hockey puck shaped separation chamber which are later described in more detail with reference to FIGS. 7-18. Separation vessel 42 is rotated or spun in the counter clockwise direction W about a vertical axis so that centrifugal force F will separate a biological fluid placed within the separation chamber of the separation vessel 42 into separate components of the biological fluid having different densities with the higher density components gravitating toward the outer circumference of the separation chamber. After separation, the biological fluid components will align in ring shaped formations from highest density to lowest density with the highest density component forming a ring shape located at the outermost circumference of the separation chamber and lower density components forming ring shapes traveling inward toward the center of the separation chamber. In the case where the biological fluid is whole blood, a red blood cell portion will form the outermost ring contained within the separation chamber followed by a ring shaped buffy coat portion which is in turn followed by a ring shaped plasma portion which is the component of the blood located nearest to the center of the separation chamber. In some instances, blood components will be separated by a cell selector gel that can form a barrier between PRP and red blood cells.

FIG. 7 is a top perspective view of the fourth exemplary embodiment of the separation vessel 42 shown in FIG. 6 with locations of separated components of whole blood in the separation chamber after separation. Separation vessel 42 includes a hollow donut-shaped/ring-shaped separation chamber 42 which is removably connected to a hollow tubular-shaped collection chamber 54. Hollow donut-shaped/ring-shaped separation chamber 42 has a top 44, a bottom 46, and a short cylindrical shaped channel 48 having a top open end 50 and a bottom open end 52 that extends from the top 44 of the separation chamber 42 to the bottom 46 of the separation chamber 42. Hollow donut-shaped/ring-shaped separation chamber 42 also includes a ring-shaped opening 53 in the bottom 46 of hollow donut-shaped/ring-shaped separation chamber 42 that surrounds short cylindrical shaped channel 48 of hollow donut-shaped/ring-shaped separation chamber 42 thereby providing an opening in hollow donut-shaped/ring-shaped separation chamber 42 through which a separated blood component, such as plasma or plasma-rich-platelets, can exit into hollow cylindrical-shaped collection tube 54. Hollow donut-shaped/ring-shaped separation chamber 42 may also include an internal chamber that can be adjusted so that the ring, disc, donut, torus can be used for various volumes of biological fluid and in some instances 1, 5, 10, 20, 30, 60, 120 mL of biological fluid.

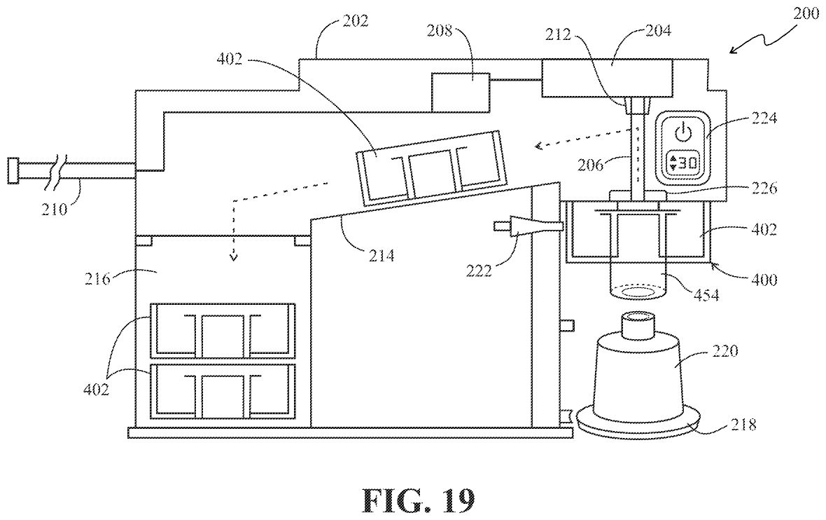

FIG. 9 is a perspective view of the tubular shaped collection chamber 54 of the separation vessel 40 shown in FIG. 7. Hollow cylindrical shaped collection chamber 54 has an open top end 56 capable of engaging with ring-shaped opening 53 in hollow donut-shaped/ring-shaped separation chamber 42 and an open bottom end 58. A rotatable locking mechanism 60 is used to lock and secure hollow cylindrical shaped collection chamber 54 to the bottom 46 of hollow donut-shaped/ring-shaped separation chamber 42 such that the ring-shaped opening 53 in the bottom of hollow donut-shaped/ring-shaped separation chamber 42 is covered in a closed position so that biological fluid inserted into the donut-shaped/ring-shaped separation chamber 42 is contained within the separation chamber 42 during rotation of the separation vessel 40. Once rotation of the separation vessel 40 stops, the locking mechanism 60 can be unlocked to enable the ring-shaped opening 53 in the bottom of hollow donut-shaped/ring-shaped separation chamber 42 to open so that a biological fluid component having a lower density that is located near the center of the separation chamber 42 after separation can exit the separation chamber 42 into the hollow tubular-shaped collection chamber 54. In the case of separating whole blood, plasma or platelet-rich plasma can exit the separation chamber 42 into the collection chamber 54. The collection chamber 54 may have a closed bottom as shown in FIGS. 10-12. Alternatively, plasma or platelet-rich plasma may pass through the open bottom end 58 of collection chamber 54 and directly into a container positioned beneath the collection chamber 54 as shown in the system and apparatus described in FIG. 19.

FIG. 8 is a schematic showing a perspective view of an exemplary embodiment of the locking mechanism 60 used to lock the hollow tubular shaped collection chamber 54 of the separation vessel 40 to the donut shaped/ring shaped separation chamber 42 of the separation vessel 40 shown in FIG. 7. Rotatable locking mechanism 60 includes a plurality of hollow notch members 62 extending from the circumference of ring-shaped opening 53 in bottom 46 of hollow donut-shaped/ring-shaped separation chamber 42 and a plurality of solid notch members 63 extending from the circumference of open top end 56 of hollow tubular shaped collection chamber 54. When the top of the hollow tubular-shaped collection chamber 54 is inserted into ring-shaped opening 53 of donut shaped/ring shaped separation chamber 42, the hollow tubular-shaped collection chamber 54 can be turned or twisted so that solid notch members 63 of the hollow tubular-shaped collection chamber 54 enter hollow notch members 62 extending from the ring-shaped opening 53 to lock the connection of the hollow tubular-shaped connection chamber 54 to the donut shaped/ring shaped separation chamber 42. After the separation of biological fluid components, the turning or twisting movement of the hollow tubular-shaped collection chamber 54 is reversed to unlock and open ring-shaped opening 53 of donut shaped/ring-shaped separation chamber 42 so that the separated component of the biological fluid positioned above the ring-shaped opening 53 in the separation chamber 42 can exit into the hollow tubular-shaped collection chamber 54. In order to isolate the separated biological fluid so that it can enter the collection chamber 54, in some exemplary embodiments the ring-shaped opening 53 or its surface area could incorporate valves, capillaries, vents, stationary phases (such as aggregated beads, membranes, etc.) and surface modifications such as hydrophilic, hydrophobic, biosensitive surfaces, and anti-fouling (biofouling) surfaces to separate the biological fluid. Alternatively, the donut-shaped/ring-shaped separation chamber 42 could collapse into a hollow cylinder for vertical extraction of the fractionated biological fluid. In addition, any combination of the preceding examples could also be utilized to separate the desired biological fluid. It will also be understood by those skilled in the art that several other types of known prior art locking mechanisms could work to removably connect and lock the separation chamber 42 to the collection chamber 54.

Hollow donut-shaped/ring-shaped separation chamber 42 may also include a female Luer-Lock fitting 65 to enable a biological fluid, such as whole blood, to be inserted into the separation chamber 42. Whole blood can be drawn from a patient into a syringe having a male Luer-Lock fitting which can then connect with the female Luer-Lock fitting 65 to expel the whole blood from the syringe into the separation chamber 42. Another alternative method for depositing blood within the separation chamber 42 may include having separation chamber 42 under vacuum so that a Vacutainer type double ended needle along with a needle holder that conforms to the shape of the separation chamber 42 can be used to pull blood from a patient through the needle and into the separation chamber 42 using the vacuum in the separation chamber 42. Separation vessel 40 may be included as part of a standard blood collection kit and/or along with a Vacutainer type double needle and needle holder that conforms to the shape of the separation chamber 42.

Alternatively, biological fluid can be placed within the ring-shaped opening 53 on the bottom 46 of the separation chamber 42 when the separation chamber is turned upside down and before the hollow tubular-shaped collection chamber is connected and locked to the separation chamber. A separator gel may also be placed within the separation chamber 42 along with the biological fluid prior to separation by rotating or spinning the separation chamber 42 to aid in forming a barrier between separated components of the biological fluid. Separation chamber 42 may be prefilled with a predetermined amount of one or more of the following: an anticoagulant, a hyaluronic acid, a selector gel including but not limited to a thixotropic gel, a biopolymer gel, a cell encapsulation ingredient (e.g. liposome, micelle) cell buffer, an anti-platelet effect buffer, an anti-microbial, an anti-bacterial, and a cell activation fluid. The cell activation fluid may include one or more of an autologous thrombin, an allogeneic thrombin, a bovine thrombin, a calcium ion, a calcium chloride, a calcium carbonate, and a calcium gluconate. The cell buffer may comprise one or more of the following and their derivatives: a glucose, a sodium ion, a sodium chloride, a magnesium ion, a magnesium chloride, a magnesium phosphate, a potassium ion, a potassium chloride, a potassium phosphate, a sodium gluconate, a pH regulator such as sodium hydroxide, and a sodium bicarbonate. The anti-platelet effect buffer may include any one or more of a honeysuckle, a thymol, an o-cymen-5-ol, a phenol, and a botanical extract.

A rod or spindle can be connected to, or inserted within, the top open end 50 of the short cylindrical shaped channel 48 of the hollow donut-shaped/ring-shaped separation chamber 42 to rotate or spin the separation vessel 40 to separate components of the biological fluid. Separation vessel 40 may be rotated in any number of ways including, but not limited to, use of a rod which spins separation vessel 40 on a vertical axis via mechanical rotation of the rod with a motor, use of forced air used to spin the separation vessel 40 on a vertical axis by securing the separation vessel 40 in a specific orientation, and use of a rotating magnetic field that spins the separation vessel 40 about a vertical axis.

Magnets may be placed externally to the separation vessel 40 and reside in a system or housing where the magnets levitate the separation vessel 40 to reduce friction. Separation vessel 40 may include internal magnets and in some instances an electromagnet where the electromagnet induces an electrical current and magnetic field on to the vessel as the vessel rotates to enhance fractionation. The magnets may be placed such that they help guide the fluid through chambers that may be contained in separation vessel 40 for further delineation of a biological fluid specimen.

FIG. 10 is a second exemplary embodiment of the tubular shaped collection chamber 74 of the separation vessel 40. Hollow tubular shaped collection chamber 74 has an open top end 76, like the open top end 56 of hollow tubular shaped collection chamber 54 shown in FIG. 9, and a closed funnel-shaped bottom end 78 having a female Luer-Lock fitting 80. Female Luer-Lock fitting 80 is capable of engaging with a male Luer-Lock fitting on a syringe 81, as shown in FIG. 11, to extract a separated component of a biological fluid from the hollow tubular shaped collection chamber 74 of the separation vessel 40. Another alternative method for extracting a separated component of a biological fluid from the hollow tubular shaped collection chamber 74 may include having collection chamber 74 under vacuum so that a Vacutainer type double ended needle along with a needle holder that conforms to the shape of the collection chamber 74 can be used to extract the separated component of the biological fluid from the collection chamber 74 through the needle and into an empty collection tube, an empty syringe, or an empty collection container.

FIG. 12 is a perspective view of a third exemplary embodiment of the hollow tubular shaped collection chamber 84 of the separation vessel 40. Hollow tubular shaped collection chamber 84 includes an open top end 86, an open bottom end 88, and a hollow funnel-shaped member 90 that can be connected to the open bottom end 88 of the hollow tubular shaped collection member 84. Hollow funnel-shaped member 90 has an open top end 92, a closed bottom end 94, and a female Luer-Lock fitting that is capable of mating with a male Luer-Lock fitting on a syringe. The open top end 92 of the hollow funnel-shaped member 90 is connected to the open bottom end 88 of the hollow tubular-shaped collection chamber 84 by a locking mechanism 100. The locking mechanism 100 includes a plurality of hollow notch members 102 extending from a circumference of the open bottom end 88 of the hollow tubular-shaped collection chamber 84 and a plurality of sold notch members 104 extending from the open top end 92 of the hollow funnel-shaped member 90. When the top of the hollow funnel-shaped member 90 is inserted into the open bottom end 88 of hollow tubular-shaped collection chamber 84, the hollow funnel-shaped member 90 can be turned or twisted so that solid notch members 104 of the hollow funnel-shaped member 90 enter hollow notch members 102 extending from the open bottom end 88 of the hollow tubular-shaped collection member 84 to lock the connection of the hollow funnel-shaped member 90 to the hollow tubular-shaped collection chamber 84. Female Luer-Lock fitting 96 is capable of engaging with a male Luer-Lock fitting on a syringe to extract a separated component, of a biological fluid from the hollow funnel-shaped member 90. Alternatively, the turning or twisting movement of the hollow funnel-shaped member 90 can be reversed to unlock and remove the hollow funnel-shaped member 90 from the hollow tubular-shaped collection chamber 84 so that a container can be placed below the open bottom end 88 of the hollow tubular-shaped collection chamber 84 to collect a separated component of the biological fluid after separation of the biological fluid. Another alternative method for extracting a separated component of a biological fluid from the hollow tubular shaped collection chamber 84 may include having collection chamber 84 under vacuum so that a Vacutainer type double ended needle along with a needle holder that conforms to the shape of the collection chamber 84 can be used to extract the separated component of the biological fluid from the collection chamber 84 through the needle and into an empty collection tube, an empty syringe, or an empty collection container.

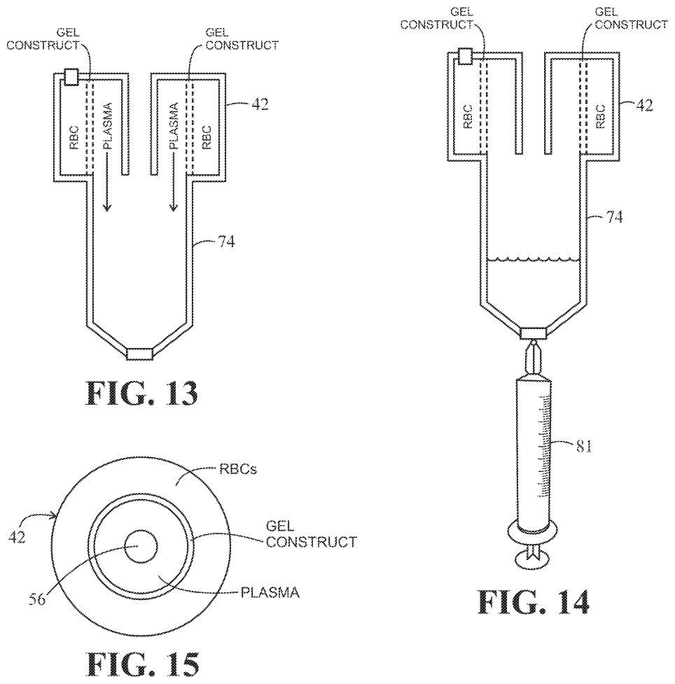

FIG. 13 is a vertical cross-sectional view of the fourth exemplary embodiment of the separation vessel 40 shown in FIG. 7 with the second exemplary embodiment of the tubular shaped collection chamber 74 shown in FIGS. 10 and 11 along with indications of where the separated fractions of blood would exist after separation of a whole blood sample utilizing the hollow donut-shaped/ring-shaped separation chamber 42 shown in FIG. 7. FIG. 14 is the same vertical cross-sectional view shown in FIG. 13 shown after the plasma portion of the separated whole blood sample falls into the tubular shaped collection chamber 74 so that it can be extracted from the tubular shaped collection chamber 74 with a syringe 81. FIG. 15 is a top plan view showing the inside of the hollow donut-shaped/ring-shaped separation chamber 42 depicted in FIG. 13 and the locations of the separated components of a whole blood sample after the whole blood sample has been separated using the hollow donut-shaped/ring-shaped separation chamber 42.

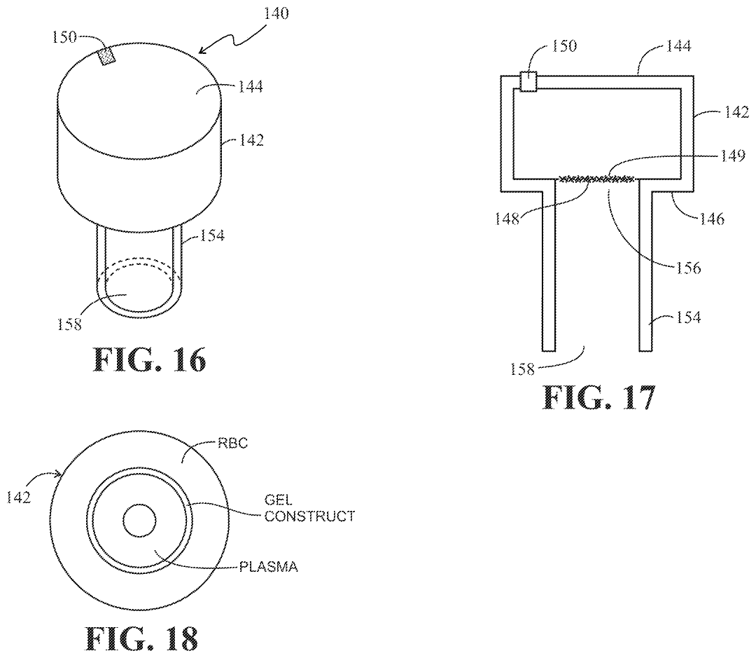

FIG. 16 is a perspective view of a fifth exemplary embodiment of a separation vessel 140 of the present invention for separating biological fluids such as whole blood into its component parts. Separation vessel 140 includes a hockey puck/disc shaped separation chamber 142 connected to a hollow tubular shaped-collection chamber 154. Hockey puck/disc shaped separation chamber 142 has a top 144, a bottom 146 with a circular shaped opening 148, and a moveable/retractable leak-proof cover member 149 which covers circular shaped opening 148. The hollow tubular-shaped collection chamber 154 has an open top end 156 and an open bottom end 158. The hollow-tubular shaped collection chamber 154 may also take the form of any of the hollow tubular shaped collections chambers previously described with reference to FIGS. 9-12. If the hollow tubular-shaped collection chamber 154 is removably connected to hockey puck/disc shaped separation chamber 142, then a biological fluid, such as whole blood, can be placed within the separation chamber 142 by turning the separation chamber 142 upside down and removing or retracting the moveable/removable cover member 149 so that the circular shaped opening 148 in the bottom 146 of the separation chamber 142 is exposed. The biological fluid, such as whole blood, can be inserted into the separation chamber 142 through the circular shaped opening 148. The moveable/retractable cover member 149 can then be moved back so that it covers the circular shaped opening 148 in the bottom of the separation chamber 142. The hollow tubular-shaped collection chamber 154 may be reconnected to the hockey puck/disc shaped separation chamber 142 prior to rotating or spinning the separation chamber 142 about a vertical axis. Components of the biological fluid contained within the separation chamber 142 are separated by rotating or spinning the separation chamber 142 about a vertical axis. Rotation or spinning of the separation chamber 142 or entire separation vessel 140 may be performed in any number of ways including, but not limited to, use of a rod on which the separation vessel 140 may be made to spin on a vertical axis via mechanical rotation of the rod with a motor, use of forced air used to spin the separation vessel 140 on a vertical axis by securing the separation vessel 140 in a specific orientation, and/or use of a rotating magnetic field that spins the separation vessel 140 about a vertical axis.

Magnets may be placed externally to the separation vessel 140 and reside in a system or housing where the magnets levitate the separation vessel 140 to reduce friction. Separation vessel 140 may include internal magnets and in some instances an electromagnet where the electromagnet induces an electrical current and magnetic field on to the vessel as the vessel rotates to enhance fractionation. The magnets may be placed such that they help guide the fluid through chambers that may be contained in separation vessel 140 for further delineation of a biological fluid specimen.

Biological fluid may also be placed within the separation chamber 142 by expelling the biological fluid contained within a syringe into the separation chamber 142 by locking a male Luer-Lock fitting on the syringe to the female Luer-Lock fitting 150 on the separation chamber 142. Another alternative method for depositing blood within the separation chamber 142 may include having separation chamber 142 under vacuum so that a Vacutainer type double ended needle along with a needle holder that conforms to the shape of the separation chamber 142 can be used to pull blood from a patient through the needle and into the separation chamber 142 using the vacuum in the separation chamber 142. Separation vessel 140 may be included as part of a standard blood collection kit and/or along with a Vacutainer type double needle and needle holder that conforms to the shape of the separation chamber 142.

FIG. 18 is a top plan view showing the inside of the separation chamber 142 depicted in FIG. 16 and the locations of the separated components of a whole blood sample after the whole blood sample has been separated using the separation chamber 142. As previously described, a separating gel may be added to the whole blood sample to assist in forming separation barriers between the separated blood components. Separation chamber 142 may be prefilled with a predetermined amount of one or more of the following: an anticoagulant, a hyaluronic acid, a selector gel including but not limited to a thixotropic gel, a biopolymer gel, a cell encapsulation ingredient (e.g. liposome, micelle) cell buffer, an anti-platelet effect buffer, an anti-microbial, an anti-bacterial, and a cell activation fluid. The cell activation fluid may include one or more of an autologous thrombin, an allogeneic thrombin, a bovine thrombin, a calcium ion, a calcium chloride, a calcium carbonate, and a calcium gluconate. The cell buffer may comprise one or more of the following and their derivatives: a glucose, a sodium ion, a sodium chloride, a magnesium ion, a magnesium chloride, a magnesium phosphate, a potassium ion, a potassium chloride, a potassium phosphate, a sodium gluconate, a pH regulator such as sodium hydroxide, and a sodium bicarbonate. The anti-platelet effect buffer may include any one or more of a honeysuckle, a thymol, an o-cymen-5-ol, a phenol, and a botanical extract.

In addition, UV light may be exposed to the separated components of the blood to convert the separating gel into a permanent barrier between blood components thereby making it easier to expel or extract plasma or platelet-rich plasma from the whole blood sample. After separation of the components of the biological fluid, the moveable/retractable cover member 149 which covers the circular shaped opening 148 in the bottom 146 of separation chamber 142 may be moved or retracted to allow a separated component of the biological fluid, such as plasma or platelet-rich plasma to fall down into hollow tubular-shaped collection chamber 154.

FIG. 19 is a cross-sectional view of a system/apparatus 200 and its components for separating biological fluids such as whole blood which utilizes the exemplary embodiment of the separation vessel shown in FIG. 7 shown along with a perspective view of part of the collection chamber 454 of the separation vessel 400 and a container 220 for collecting platelet rich plasma from the collection chamber 454 of the separation vessel 400 seated on an adjustable tray 218. System/apparatus 200 includes a housing 202 that contains a motor 204 connected to a spindle/rod 206 for rotating a separation vessel 400 capable of being connected to the spindle/rod 206, an actuator 212 for enabling vertical movement and release of hollow separation chamber 402 from hollow tubular-shaped collection chamber 454, a printed circuit board 208 for connecting the motor 204 and actuator 212 to a power source and for programming and operating the system/apparatus 200, an electrical cord 210 for connecting the system/apparatus to a power source, a biohazard collection bin 216 for retaining used separation chambers 402, a ramp/pathway 214 for directing movement of used separation chambers 402 into the biohazard collection bin 216, a laser 222 to read radio-frequency identification labels, an ultraviolet (UV) light source 226 for exposing the separated components of a biological fluid contained within the separation chamber 402 to UV light, and a user interface 224 for activating and programming the system/apparatus 200. The system/apparatus 200 may also include an adjustable tray 218 connectable to housing 200 for retaining or holding a container 220 for containing a separated component of a biological fluid such as plasma or platelet-rich plasma.

The system/apparatus 200 may be capable of automatically optimizing centrifugal force, angular speed, pressure applied to the external separation vessel, and air flow for enhanced fractionation. The system/apparatus 200 may also be capable of adjusting for multiple sizes of separation vessels. System/apparatus 200 may also enable a fraction of the biological fluid or tissue to be expelled and aspirated into another vessel for a second rotation step. The second rotation would produce a pellet of cells on the outside of the separation vessel (e.g. a pellet of cells on the outside of the hollow donut-shaped/ring-shaped separation vessel) with a supernatant filling the rest of the separation vessel. System/apparatus 200 may then further enable a portion of the supernatant to be extracted and the remainder of the supernatant to be mixed with the pellet of cells via agitation. The system/apparatus 200 may also include an automated cell counter which in some cases may display a patient's platelet count.

The system/apparatus 200 may further include a means for inducing pressure for blood extraction from a patient and means for optimizing the pressure to ensure vein integrity. System/apparatus 200 may also include a vacuum and a cannula connectable to the vacuum for enabling the removal of fat deposits from a patient. System/apparatus may also include means for optimizing the separation or mixing of adipose tissue.

System and apparatus 200 may be used to separate biological tissue. For example, the fat in adipose tissue can be centrifuged resulting in a density gradient where in some cases Adipose-derived stem cells (ASCs) are concentrated on the bottom of the cylinder if a cylinder shape is used for the separation vessel or separation chamber. This fraction is called the stromal vascular fraction (SVF) where in some methods the system has the potential to isolate stromal vascular cells (SVCs).

In addition to separating biological fluids, the system/apparatus 200 is capable of mixing at least one of a biological tissue and a biological fluid with other materials to provide a composition that includes the biological tissue and/or biological fluid in a cosmetically acceptable carrier or a pharmaceutically acceptable carrier. One example includes mixing a biological tissue and/or biological fluid in a gel, such as an optimized hydrogel that is capable of cross-linking with the biological tissue and/or biological fluid to create a pharmaceutical grade aesthetic injectable such as a filler for cosmetic volume enhancement.

The drawings and description of exemplary embodiments of the invention herein shows various exemplary embodiments of the invention. These exemplary embodiments and modes are described in sufficient detail to enable those skilled in the art to practice the invention and are not intended to limit the scope, applicability, or configuration of the invention in any way. Rather, the following disclosure is intended to teach both the implementation of the exemplary embodiments and modes and any equivalent modes or embodiments that are known or obvious to those reasonably skilled in the art. Additionally, all included examples are non-limiting illustrations of the exemplary embodiments and modes, which similarly avail themselves to any equivalent modes or embodiments that are known or obvious to those reasonably skilled in the art.

Other combinations and/or modifications of structures, arrangements, applications, proportions, elements, materials, or components used in the practice of the instant invention, in addition to those not specifically recited, can be varied or otherwise particularly adapted to specific environments, manufacturing specifications, design parameters, or other operating requirements without departing from the scope of the instant invention and are intended to be included in this disclosure.

Unless specifically noted, it is the Applicant's intent that the words and phrases in the specification and the claims be given the commonly accepted generic meaning or an ordinary and accustomed meaning used by those of ordinary skill in the applicable arts. In the instance where these meanings differ, the words and phrases in the specification and the claims should be given the broadest possible, generic meaning. If any other special meaning is intended for any word or phrase, the specification will clearly state and define the special meaning.

1. A device for separating biological tissue and/or biological fluids or mixing biological tissue and/or biological fluids with other materials where the device comprises a hollow sphere shaped vessel having an access port contained within a surface of the vessel.

2. The device of claim 1 further comprising a housing for retaining the hollow sphere shaped vessel and at least one air inlet positioned within the housing so that the hollow sphere shaped vessel can spin in a counterclockwise direction on a vertical axis when air is provided through the air inlet.

3. The device of claim 1 further comprising a hollow cylindrical member extending into an interior of the hollow sphere shaped vessel such that the hollow cylindrical member is contiguous with an exterior surface of the hollow sphere shaped vessel.

4. The device of claim 1 wherein the hollow sphere shaped vessel is prefilled with a predetermined amount of at least one of an anticoagulant, a hyaluronic acid, a cell selector gel, a biopolymer gel, a cell encapsulation cell buffer, an anti-platelet effect buffer, an anti-microbial, and anti-bacterial, and a cell activation fluid.

5. The device of claim 1 wherein the hollow sphere shaped vessel is under vacuum and is compatible with a double ended needle and a needle holder that conforms to at least a portion of the shape of the hollow sphere shaped vessel.

6. A device for separating biological tissue and/or biological fluids or mixing biological tissue and/or biological fluids with other materials where the device comprises a hollow cylindrical shaped vessel having an access port contained within a surface of the vessel.

7. The device of claim 6 further comprising a housing for retaining the hollow cylindrical shaped vessel and at least one air inlet positioned within the housing so that the hollow cylindrical shaped vessel can spin in a counterclockwise direction on a vertical axis when air is provided through the air inlet.

8. The device of claim 6 further comprising a second smaller hollow cylindrical shaped member extending into an interior of the hollow cylindrical shaped vessel such that the second smaller hollow cylindrical shaped member is contiguous with an exterior surface of the hollow cylindrical shaped vessel.

9. The device of claim 6 wherein the hollow cylindrical shaped vessel is prefilled with a predetermined amount of at least one of an anticoagulant, a hyaluronic acid, a cell selector gel, a biopolymer gel, a cell encapsulation cell buffer, an anti-platelet effect buffer, an anti-microbial, and anti-bacterial, and a cell activation fluid.

10. The device of claim 6 wherein the hollow cylindrical shaped vessel is under vacuum and is compatible with a double ended needle and a needle holder that conforms to at least a portion of the shape of the hollow cylindrical shaped vessel.

11. A vessel for separating biological tissue and/or biological fluids or mixing biological tissue and/or biological fluids with other materials where the device comprises a hollow hockey puck shaped chamber having a circular shaped opening in a bottom of the chamber and a hollow tubular shaped collection chamber having a top open end wherein the top open end of the collection chamber is in communication with the circular shaped opening in the bottom of the hollow hockey puck shaped chamber.

12. The vessel of claim 11 further comprising a moveable or retractable cover member capable of covering the circular shaped opening in the bottom of the hollow hockey puck shaped chamber.

13. The vessel of claim 11 further comprising an access port contained within a surface of the hollow hockey puck shaped chamber.

14. The vessel of claim 11 wherein the hollow hockey puck shaped chamber is prefilled with a predetermined amount of at least one of an anticoagulant, a hyaluronic acid, a cell selector gel, a biopolymer gel, a cell encapsulation cell buffer, an anti-platelet effect buffer, an anti-microbial, and anti-bacterial, and a cell activation fluid.

15. The vessel of claim 11 wherein the hollow hockey puck shaped shaped chamber is under vacuum and is compatible with a double ended needle and a needle holder that conforms to at least a portion of the shape of the hollow hockey puck shaped chamber.

16. A vessel for separating biological tissue and/or biological fluids or mixing biological tissue and/or biological fluids with other materials where the vessel comprises a hollow donut-shaped/ring-shaped chamber having a ring shaped opening contained in a bottom of the hollow donut-shaped/ring-shaped chamber and a hollow tubular-shaped collection chamber having an open top end wherein the open top end of the collection chamber is in communication with the ring shaped opening in the hollow donut-shaped/ring-shaped chamber.

17. The vessel of claim 16 further comprising an access port contained within a surface of the hollow donut-shaped/ring-shaped chamber.

18. The vessel of claim 16 wherein the hollow tubular-shaped collection chamber includes a closed funnel shaped bottom having an access port.

19. The separation vessel of claim 18 wherein the closed, funnel shaped bottom of the collection chamber is removable.

20. An apparatus for separating biological tissue and/or biological fluids or mixing biological tissue and/or biological fluids with other materials comprising:
a housing;
a spindle/rod member contained within the housing;
a motor contained within the housing capable of rotating the spindle/rod member;
a vessel having an upper hollow chamber in communication with a lower hollow tubular-shaped collection chamber wherein the spindle/rod member is capable of engaging with the upper hollow chamber of the vessel to rotate/spin the vessel about a vertical axis.

21. The apparatus of claim 20 further comprising a user interface to select at least one of a rotation speed and time for rotating/spinning the vessel.

22. The apparatus of claim 20 further comprising a biohazard collection bin contained, within the housing and a ramp/pathway contained within the housing for directing used hollow chambers of the vessel into the biohazard collection bin.

23. The apparatus of claim 20 further comprising an adjustable tray for retaining a container for containing a separated biological fluid or a biological tissue and/or biological fluid mixed with another material.

24. The apparatus of claim 20 further comprising an ultraviolet light source contained within the housing for exposing separated components of a biological fluid contained within the hollow chamber to ultraviolet light.
